(19) United States
(12) Patent Application Publication
(10) Pub. No.: US 2008/0051816 A1
(43) Pub. Date: Feb. 28, 2008

(54) HAIR IMPLANT APPARATUS AND METHOD FOR ITS USE

(76) Inventors: Jae P. Pak, (US); William R. Rassman, (US)

Correspondence Address: Jae P. Pak, M.D., Suite 301, 9911 W. Pico Blvd., Los Angeles, CA, 90035, US

(21) Appl. No.: 11/466,359

(22) Filed: Aug. 22, 2006

(51) Int. Cl. A61B 17/34

(52) U.S. Cl. 606/187

(57) ABSTRACT

Apparatus for implanting single hair grafts in implant sites in a skin is adapted to apply a tiny adhesive dot in a manner to secure the implanted graft to the skin surface. The quick hardening of the adhesive allows the placement of implants in closely spaced, neighboring sites without causing dislodgment of previously implanted grafts. Illustrative apparatus for providing such implants comprises a hollow needle with a coaxial solid rod in a fixed position. The needle is moved along its axis for implanting a graft and a channel in the rod is positioned to deliver the adhesive dot.

### FIELD OF THE INVENTION

This invention relates to apparatus for implanting a single hair graft or follicular unit into an implant site in skin and more particularly to an apparatus and method for producing securely attached implants.

### BACKGROUND OF THE INVENTION

Many devices are known in the art each of which is operative for implanting hair grafts. Copending application Ser. No. 10,397,685 filed Mar. 26, 2003 discloses one such implant device which comprises a hollow needle movable, under user control, along its center axis and a rod positioned along the center axis. The rod is in a fixed position and secured to the housing of the device. The needle has a side aperture adapted to supply a hair graft for implant.

The needle is moved between a first and a second position with respect to the fixed rod such that the side aperture is at the distal end of the rod and the distal end of the needle is at the distal end of the rod, respectively. Thus, when the side aperture is at the end of the rod, the needle is inserted in an implant site and a hair graft is located at the rod end. When the needle is withdrawn with respect to the rod, the rod end is at the skin surface and the graft is retained in the skin by the rod.

As is seemingly the case with prior art devices for implanting a sequence of follicular units or hair grafts, the close placement of consecutive grafts often causes dislodgment of previously, closely spaced implants. This dislodgment, of course, necessitates close inspection and frequent repetition of an implanting process.

### SUMMARY

It has been found that an adhesive dot can be applied in a manner to secure a graft which is placed in an implant site and that the adhesive can be hardened sufficiently quickly to allow prompt placements of implants in closely spaced neighboring sites. Although adhesives have been used to secure hair plugs in the past, the use of adhesives had been discontinued because (1) the adhesives became stringy and sticky and was not useful in securing the hair grafts in the large scalp areas incised to accept it and (2) because the standard of care has moved to very small grafts, which tend to remain in position if the recipient site has been made well in advance of placing the grafts. When the graft recipient site is made at about the same time or immediately prior to inserting the graft, there is pressure created from making the wound causing the adjacent grafts to extrude. This has been a standard limitation to the percutaneous placement of hair grafts with implanting instruments.

The placement of a tiny amount of adhesive at a correct time in a percutaneous implant of a single graft, and the choice of adhesive and hardening properties was found not only to be advantageous, but also to be highly controllable and capable of being hardened sufficiently quickly to allow implants to be placed in closely spaced neighboring sites in a timely fashion.

### BRIEF DESCRIPTION OF THE DRAWINGS

FIG. 1 is a schematic representation, partially in cross section of a prior art graft implanter adaptable in accordance with the principles of this invention;

FIG. 2 is a schematic block diagram, partially in cross section showing a graft implanter of the type shown in FIG. 1 adapted for operation in accordance with the principles of this invention;

FIGS. 3-5 are schematic illustrations of the sequence of positions of the components of the implanter of FIG. 2 during operations; and

FIGS. 6-8 are schematic illustrations of the sequence of positions of components of an alternative embodiment in accordance with the principles of this invention.

### DETAILED DESCRIPTION OF ILLUSTRATIVE EMBODIMENTS OF THIS INVENTION

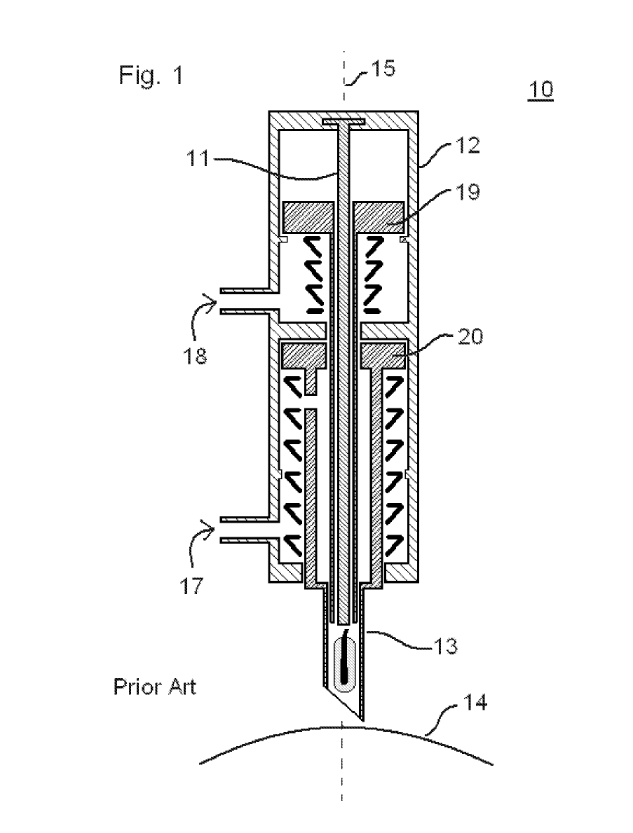

FIG. 1 is a schematic representation, partially in cross section of apparatus 10 adaptable in accordance with the principles of this invention. The apparatus comprises an elongated rod 11 secured to the housing 12 in a fixed position during operation of the apparatus.

The apparatus also comprises a sheath 13 which is of reduced dimension to form a cutting edge such as a needle point (or a punch) at its distal end which is directed at a skin area indicated at 14. The sheath is hollow as is clear from the figure and forms a hollow needle which has an axis with which rod 11 is aligned. The axis of the needle and rod is indicated by dashed line 15

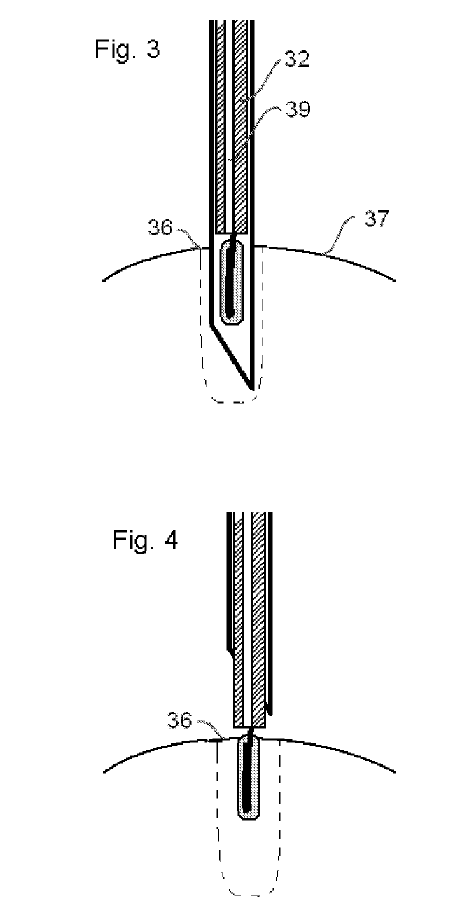

Operation of the apparatus is in response to vacuums applied in the alternative or in concert at opening 17 and 18 in housing 12. The vacuums move pistons 19 and 20 in a manner to move the hollow needle into a sequence of positions described in connection with FIGS. 3 through 6.

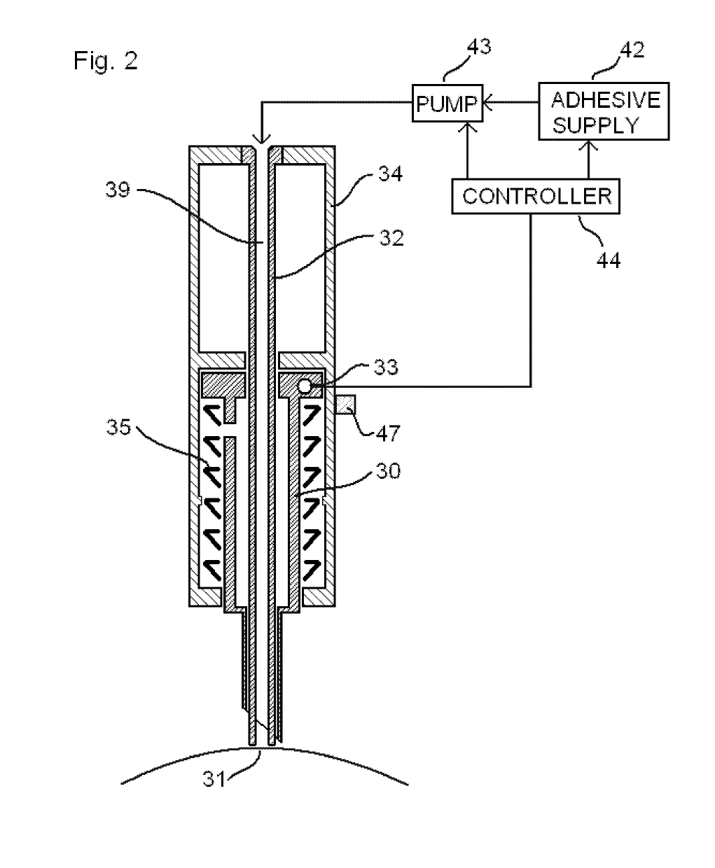

The operation of the apparatus in response to vacuum inputs is fully described in the above-noted patent application and lends itself to robotic operation under automated control. But the entire operation can be operated manually from an initial positioning of the apparatus as shown in FIG. 2. The operation depends on the movement of sheath 30, (and thus the hollow needle) from position aligned with the distal end 31 of rod 32. Manual operation is achieved by the movement downward, as view in FIG. 2, of piston 30 by a handle indicated at 33. The movement of the handle is along a slot (not shown) in housing 34. The sheath is returned to the position shown in FIG. 2 by springs 35.

FIG. 3 shows the position of the sheath (or hollow needle) partially inside an implant site 36 in skin 37, a position achieved by the lowering of sheath 30. FIG. 2 also shows a channel 39 in rod 32 for the delivery of adhesive to the area where a graft touches the skin surface at an implant site.

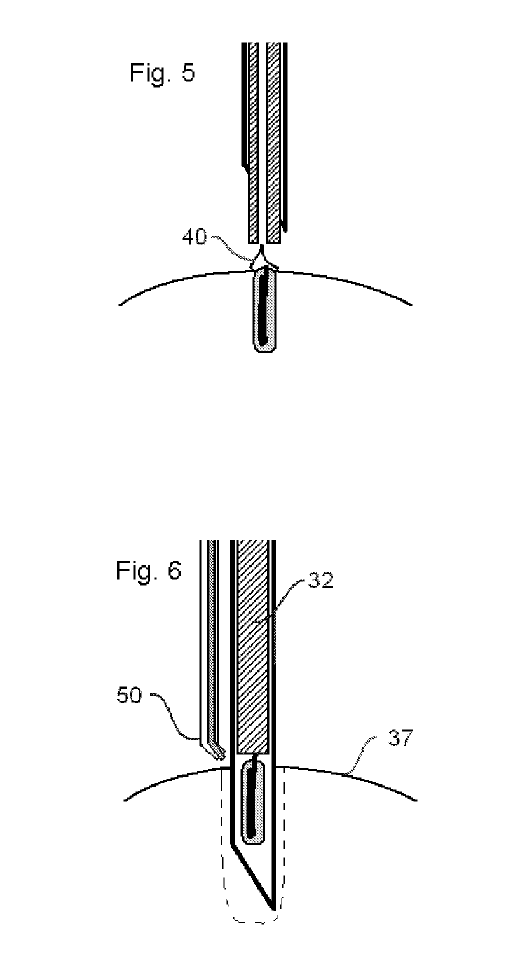

FIG. 4 shows the needle in a withdrawn position with the rod remaining in place closely abutting the implant site. The withdrawal of the needle is achieved illustratively by the release of handle 33 and the action of spring 35 of FIG. 2. An adhesive dot 40 is conveniently applied at this juncture in the operation as indicated in FIG. 5.

Channel 39 is coupled to an adhesive reservoir 42 via a pump 43 activated under the control of user-operated (or preprogrammed) controller 44 as shown in FIG. 2. The delivery of an adhesive dot may be timed to the insertion of the needle into an implant site or to the removal of the needle from the implant site. The timing is conveniently responsive to a signal from a sensor 47, shown in FIG. 2, indicating the position of the sheath (or hollow needle).

FIGS. 2-5 relate to apparatus which includes an adhesive channel within rod 32. FIGS. 6 through 9 relate to an embodiment of this invention where the adhesive delivery system has a conduit 50 external to the rod (32) and may even be external to the housing of the apparatus.

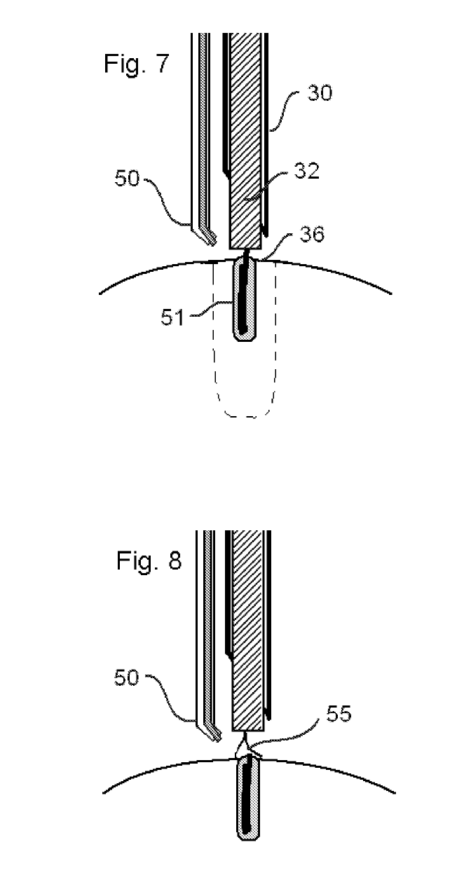

With specific reference to FIG. 6, the rod (32) and the needle (30) are in an initial position as also shown in FIG. 3. FIG. 6 also shows an adhesive conduit 50. FIG. 7 corresponds to FIG. 4 showing a hair graft 51 positioned within an implant site and FIG. 8 shows the needle withdrawn from the implant site with the rod and the adhesive conduit properly positioned to deliver an adhesive dot.

FIG. 8 shows adhesive dot 55 properly positioned as the rod is removed from a position abutting the skin.

A variety of adhesives lend themselves for use in accordance with the principles of this invention. Adhesives illustratively found useful include 2-Octyl Cyanoacrylate or topical thrombin or clotting factor or starch based adhesives.

The quantity of adhesive supplied in each instance as well as the time required for curing of the adhesive determines the window for the placement of another near-neighbor implant.

As is clear from the foregoing discussion, ultra violet hardening of UV responsive adhesive permits very fast hardening to be achieved. But other substances with adhesive like properties harden on contact and others air dry relatively quickly. Such is the case with Biological implantable adhesives which may be placed in the wound and activate upon contact with biological wounds. These adhesives have a different mechanism and timing for placement.

What has been described herein is merely illustrative of the principles of this invention and it is clearly within the purview of one skilled in the art to produce variations thereof within the spirit and scope of the invention as encompassed by the following claims. For example, vacuum and spring loaded activation have been described herein. But compressed air or electric solenoids may also be used. Also, implant site may be prepared prior to the insertion of a graft herein as well as being formed by the advancement of the needle as described herein, and the graft may even be inserted by tweezers.

What is claimed is:

1. Apparatus for implanting a hair graft into skin, said apparatus comprising a sheath with a cutting edge at its distal end and having an axis, said apparatus including a rod along said axis, said sheath being movable with respect to said rod in a manner to expel a hair graft from the distal end of said sheath, said apparatus also including an adhesive delivery system adapted to administer adhesive at a time and in a position to secure a hair graft to an implant site in said skin.

2. Apparatus as in claim 1 wherein said adhesive delivery system is adapted to administer a microdot of adhesive at a time when said hair graft is being placed in an implant site.

3. Apparatus as in claim 1 wherein said rod is in a fixed position and a hair graft is placed in an implant site by inserting the distal end of said sheath into the implant site and withdrawing said sheath with respect to said rod to expel said graft and retain the graft in place while the sheath is withdrawn, wherein the adhesive is administered at a user-selected time corresponding to the insertion of the proximal end of said sheath into an implant site, the withdrawal of the proximal end from the implant site, the withdrawal of the rod from the implant site or to the hair graft itself just prior the movement of said rod to expel said hair graft into the selected implant site.

4. Apparatus as in claim 1 wherein said adhesive delivery system includes an adhesive delivery channel formed in said rod and adapted to apply an adhesive dot to a hair graft at a time when said rod is withdrawn from an implant site and a hair graft is retained in the site.

5. Apparatus as in claim 4 wherein said adhesive delivery system includes an adhesive delivery conduit external to said sheath and positioned in a manner to deliver said adhesive.

6. Apparatus as in claim 4 including a pump coupled to said channel, said pump being connected to an adhesive reservoir and operative to pulse adhesive in said channel in response to a user-selected command timed to the position of said sheath.

7. Apparatus as in claim 3 wherein said sheath has a side aperture and said sheath is moved under user control between first and second positions such that the side aperture is located at the proximal end of said rod and at the proximal end of the sheath respectively.

8. A method of implanting hair grafts at implant sites in a skin, said method comprising the steps of inserting a hair graft into a selected one of said implant sites, and applying an adhesive dot in a manner to secure said hair graft to the surface of said skin at said site.

9. A method as in claim 8 also including the step of curing or hardening said adhesive dot.

10. A method as in claim 9 wherein said adhesive is selected from a group of adhesives consisting of cyanoacrylate family and said curing is accomplished by U.V. light.

11. A method as in claim 10 wherein a hair graft is inserted into each of a sequence of neighboring implant sites and said hardening is completed in a time prior to the insertion of a next hair graft in a neighboring implant site.

12. A method as in claim 8 wherein said adhesive dot comprises of a starch based adhesive

13. A method as in claim 8 wherein said adhesive dot comprises of a topical thrombin or clotting factor adhesive.

14. Apparatus for implanting a hair graft, said apparatus comprising means for inserting a graft into an implant site and means for applying an adhesive to the graft in a manner to secure the graft in place.
